(19) United States
(12) Patent Application Publication
(10) Pub. No.: US 2022/0084671 A1
(43) Pub. Date: Mar. 17, 2022

(54) METHOD AND SYSTEM FOR DATA TRANSFER IN VENTILATORS

(71) Applicant: Loewenstein Medical Technology S.A., Luxembourg (LU)

(72) Inventor: Matthias Schwaibold, Karlsruhe (DE)

(21) Appl. No.: 17/447,597

(22) Filed: Sep. 14, 2021

(30) Foreign Application Priority Data
Sep. 15, 2020 (DE) 102020123923.8

(51) Int. Cl. G16H 40/67

(52) U.S. Cl. CPC G16H 40/67; G16H 20/40

(57) ABSTRACT

A method for data transfer within a system, wherein the system comprises at least one ventilator and a server, the ventilator comprising at least one input unit and at least one communication unit, the input unit being set up and configured to provide input values and information to the system and the communication unit being set up and configured to make at least one connection to at least one server and to transfer data between the server and the ventilator. The data transfer is terminated by the server, the termination of the data transfer being characterized in that the ventilator transfers no further medical data to the server.

### CROSS-REFERENCE TO RELATED APPLICATIONS

The present application claims priority under 35 U.S.C. § 119 of German Patent Application No. 102020123923.8, filed Sep. 15, 2020, the entire disclosure of which is expressly incorporated by reference herein.

### BACKGROUND OF THE INVENTION

### 1. Field of the Invention

The invention relates to a method for data transfer within a system comprising at least one ventilator.

### 2. Discussion of Background Information

Modern ventilators usually have multiple options for displaying or outputting data or information concerning the user. As such, a ventilator generally has a display by way of which the information can be presented to a certain extent directly on the device. Furthermore, ventilators are known that have various options for transferring data between the ventilator and external devices.

In the prior art, these options are known in the form of connections for storage devices or perhaps connections to servers in order to transfer the data from the ventilator to other devices and to display said data thereon or to process said data (further). In particular connecting the ventilator to a server allows remote monitoring of the user, so-called telemonitoring. Telemonitoring also allows a plurality of ventilators to be monitored.

The known ventilators are constantly connected to servers for the purpose of telemonitoring and interchange data and information in this case. This leads to data being transferred and stored even at times when there is no need for transfer. As a result, unnecessary energy costs are incurred and possibly also mobile radio costs in the case of devices used on a mobile basis. Constant data transfer without necessity must be regarded as critical in respect of data protection too.

In view of the forgoing, it would be advantageous to have available a system for data transfer and a corresponding method that can lower energy consumption and simplify the monitoring of multiple ventilators.

### SUMMARY OF THE INVENTION

The present invention provides a method for data transfer within a system, wherein the system comprises at least one ventilator and a server. The ventilator comprises at least one input unit and at least one communication unit, wherein the input unit is set up and designed to provide input values and information to the system and wherein the communication unit is set up and designed to make at least one connection to at least one server and to transfer data between the server and the ventilator, that is to say to transmit data to the server and to receive data from the server. The data transfer is terminated by the server, the termination of the data transfer being characterized in that the ventilator transfers no further medical data to the server. Termination of the data transfer by the server can also arise as a result of the server transmitting an appropriate command or request to the ventilator. This should also be understood to mean that the server transmits a requirement for a volume of data and/or transmission length to the ventilator, and the ventilator terminates the data transfer following transfer of the requested data. The ventilator thus does not decide independently whether the data transfer is terminated, but rather only after a request and/or advice (such as, e.g., a volume of data to be transferred or a period for the data transfer) by the server.

In some embodiments of the method, non-medical data are transferred between the server and the ventilator independently of the data transfer.

In some embodiments of the method, non-medical data are transferred between the server and the ventilator following termination of the data transfer by the server.

In some embodiments of the method, the server transfers data to the ventilator following the termination of the data transfer by the server.

In some embodiments of the method, the connection between the server and the ventilator is broken following termination of the data transfer between the server and the ventilator.

In some embodiments of the method, the connection between the ventilator and the server is made exclusively by the communication unit. The making of a connection between the ventilator and the server can for example also be triggered by an appropriate input on the input unit.

In some embodiments of the method, a broken connection results in the communication unit connecting to the server in periods of time.

In some embodiments of the method, the connection is made only when the ventilator is in a connection-compatible mode.

In some embodiments of the method, initially only device information of the ventilator is transferred to the server when the communication unit of the ventilator has set up a connection to the server again following a period of time.

In some embodiments of the method, the server uses the transferred device information of the ventilator to identify the ventilator and decides whether and which data are supposed to be transferred from the ventilator to the server.

In some embodiments of the method, prior to terminating the data transfer and/or prior to breaking the connection the server transmits at least data concerning the period of time in which the ventilator connects to the server.

In some embodiments of the method, the ventilator automatically determines the period of time for the next connection to the server on the basis of an analysis of the previously transferred data.

In some embodiments of the method, the server transfers configuration data for communication to the ventilator, the configuration data for communication comprising settings relating to the volume of data that is supposed to be transmitted from the ventilator to the server during the data transfer and/or the connection and settings relating to the intervals of time at which the ventilator is supposed to connect to the server.

In some embodiments of the method, no medical data are transferred from the ventilator to the server if these data are not requested by the server.

In some embodiments of the method, the transfer of the data between the ventilator and the server is begun by an action command from the server to the communication unit. This action command conveys to the communication unit or the ventilator the requirement for a data transfer, for example.

In some embodiments of the method, the communication unit and/or the server generate an alarm signal or trigger an alarm signal if a specific number of connections of the communication unit to the server that have not taken place or not been successful in the predefined periods of time are registered.

In some embodiments of the method, the data transfer from the communication unit to the server is automatically terminated by the server on the basis of an analysis of the medical data previously transferred by the ventilator.

In some embodiments of the method, the connection of the communication unit to the server is automatically broken by the server on the basis of an analysis of the previously transferred data.

In some embodiments of the method, the data transfer from the communication unit to the server is terminated by the server following an input on the server.

In some embodiments of the method, the connection of the communication unit to the server is broken by the server following an input on the server.

In some embodiments of the method, data are recorded by the ventilator and are stored until the next connection to the server. In some embodiments, the recorded data are also stored following a connection to the server and/or a transfer of the stored data to the server.

In some embodiments of the method, a broken connection to the server results in the ventilator making a connection to the server on the basis of an analysis of the recorded data and initiating a data transfer.

In some embodiments of the method, the ventilator evaluates the stored data and/or combines said data. These evaluated or combined data are transferred to the server in the course of a new transfer of data.

In some embodiments of the method, the ventilator evaluates the stored data and/or combines said data. These evaluated or combined data are transferred to the server in the course of a new transfer of data, if they are requested by the server.

In some embodiments of the method, the configuration data for communication comprise settings regarding whether data having a high level of detail are supposed to be transferred.

In some embodiments of the method, the configuration data for communication comprise settings regarding whether exclusively rated and/or combined data are supposed to be transferred.

In some embodiments of the method, the ventilator connects to the server at certain intervals of time, and a data transfer takes place between the server and the ventilator.

In some embodiments of the method, the transfer of medical data is prevented after the data transfer has been terminated by the server.

The claimed apparatus relates to a ventilator that is used in the described method according to the invention.

It should be pointed out that the features individually presented in the claims can be combined with one another in any technically meaningful manner and show further configurations of the invention. The description characterizes and specifies the invention additionally in particular in connection with figures.

It will also be pointed out that an “and/or” conjunction used in this document, positioned between two features and linking said features to one another, should always be interpreted to mean that in a first configuration of the subject matter according to the invention only the first feature can be present, in a second configuration only the second feature can be present and in a third configuration both the first and the second feature can be present.

A ventilator should be understood to mean any device that assists a user or patient with natural breathing, undertakes ventilation of the user or patient and/or is used for respiratory therapy and/or otherwise influences the breathing of the user or patient. This includes, for example, but not exclusively, CPAP and BiPAP machines, anesthesia machines, respiratory therapy machines, (clinical, nonclinical or emergency) ventilators, high flow therapy machines and cough assist machines. Ventilators can also be understood as diagnostic devices for ventilation. Diagnostic devices can be used generally in this case to capture medical parameters of a patient. They also include devices that can capture and optionally process medical parameters from patients in combination with or relating exclusively to breathing.

An interface should be understood in the broadest sense to mean any type of device for connection—regardless of the type of connection—between the ventilator and a remote station or a person. An interface for a connection between the ventilator and a person can be a user interface, for example, which allows interaction between the person and the ventilator directly at the ventilator.

A patient interface can be understood, unless expressly described otherwise, to mean any part or connected peripheral devices of the ventilator that is intended for interaction, in particular for therapeutic or diagnostic purposes, with a patient. In particular, a patient interface can be understood to mean a mask of a ventilator or a mask connected to the ventilator. This mask can be a full face mask, that is to say covering the nose and the mouth, or a nasal mask, that is to say a mask covering only the nose. Tracheal tubes and so-called nasal cannulae can also be used as a mask.

If for example the data transfer between the ventilator and the server has been terminated and, as a result, the connection between the ventilator and the server has also been broken, it is possible to set the ventilator to the effect that a broken connection between the ventilator and the server results in the communication unit making a connection to the server, or making a connection attempt, in a specific period of time that for example is stipulated on the ventilator and/or is predefined by the server. In some embodiments, the connection is made only when the ventilator is capable of a connection and data transfer. To that end, for example at least one power supply and a data connection (for example mobile radio or Internet connection via LAN/WLAN) should be present. An active wireless network connection (for example mobile radio, WLAN/Wi-Fi) is sometimes not desirable while sleep therapy is ongoing. If a wired network connection or Internet connection is not available, it would not be possible to make a connection from the ventilator to the server in this case.

The connection and a data transfer between the ventilator and the server are made on a regular basis. To that end, the ventilator makes a connection to the server as soon as a previously defined period of time has elapsed and the ventilator is capable thereof, that is to say a power supply and data connection are available, for example. The making of a connection between the ventilator and the server can alternatively also be triggered manually by a user at the ventilator or a user of the server.

During the data transfer, the server transmits configuration data to the therapy device, said configuration data containing medical configuration data (for example therapeutic setting values), technical configuration data (for example firmware update) and configuration data for communication. The configuration data for communication contain settings relating to the volume of data that the ventilator is supposed to transmit to the server during the data transfer, and preferably also relating to the period of time. The configuration for communication can, further, also be used to set for example the level of detail with which the medical and technical data of the ventilator are supposed to be transmitted to the server.

During the data transfer, the therapy device transmits technical data (for example version, settings, fill level or wear on consumables, occurrence of technical faults, etc.) and medical data (times of use of the ventilator, effectiveness of the ventilator/treatment, correct application of the ventilator, diagnostic data from the ventilator, pressure, flow, respiratory rate, volumes, O₂/CO₂ saturation/content of (exhaled) respiratory air, etc.) to the server.

The configuration data for communication can be used by the server, or a user of the server, to remotely set in particular whether, with which period of time and with which level of detail medical data are supposed to be transmitted from the ventilator to the server. The medical data can be transmitted with different levels of detail. The level of detail for the transmission can be predefined by the server. The level of detail for the transmission can also be set by a user of the server remotely. In some embodiments, the therapy device itself can also predefine the level of detail. The data can be measured values and/or result values, for example. The data can also be transmitted in the form of signal curves. The data or values can be transmitted as raw data or as characteristic quantities such as for example mean values, medians or the like. The resolution of the data can likewise be predefined by the server. The data can be transmitted with low, medium or high resolution.

The configuration data for communication can also contain settings for whether a persistent data transfer, that is to say a constant transmission of data picked up, or a transfer of data packets, for example combined medical data, is supposed to take place. In respect of a persistent data transfer, it is for example additionally possible to set the frequency with which the data are transferred, that is to say whether a live transfer or a lower frequency (for example a transfer every 1 to 60 minutes) is supposed to take place.

In this case, each data transfer or connection can result in new configuration data for communication being transmitted from the server to the ventilator, which uses said configuration data for current and/or future connections or data transfers.

In some embodiments of the method, the ventilator initially transmits device information, containing information such as device type, serial number, firmware version, etc., to the server for each newly made connection to the server. On the basis of this information, the server initially identifies the device and can then individually decide for this device whether and what volume of data is supposed to be transmitted from the ventilator to the server. This decision is transmitted from the server to the ventilator as a configuration for communication, for example. On the basis of this configuration for communication, the ventilator begins the data transfer if necessary. The server also decides whether and which other data and/or configurations, such as for example technical or medical configurations, are transmitted to the ventilator. For example, the server can use the device information of the ventilator to decide that a new firmware version is supposed to be transmitted to the ventilator.

In some embodiments of the method, the ventilator also receives, on the basis of the configuration for communication, the period of time after which it is supposed to connect to the server again as soon as the data transfer has concluded. Alternatively, the ventilator can also be set up to determine the period of time on the basis of the transferred data themselves. If for example medical data have just been requested and transmitted from the ventilator to the server, then the next connection from the ventilator to the server takes place after a period of time of 24 hours, for example. If no medical data have been requested, the period of time can also be 3 or 5 or 7 days, for example.

After the period of time for the next connection of the ventilator to the server has elapsed, both the ventilator and the server register whether a connection has been made successfully. In some embodiments of the method, the server counts the number of connections that have not been made, for example, and after a specific number of missing connections an alarm notification is generated that is output via the server and displayed to a user of the server. Alternatively or additionally, the server can also be set such that a connection to the ventilator by the server is sought should a number of connections that have not been made be registered. The number of connections not made between the ventilator and the server that leads to the generation of an alarm notification is dependent on the previously set period of time and the last data transfer, inter alia. If for example the set period of time is 24 hours, then an alarm notification can be generated by the server after just 2 connections that have not been made (that is to say 48 hours). A threshold of just one connection that has not been made or 3 or more connections that have not been made is also conceivable and can be useful. If the last data transfer was a persistent, that is to say “live”, transfer or a transfer of data at short intervals of time (frequency of one data transfer every 1 to 360 minutes), then it can be useful to select the number of connections not made that lead to an alarm notification to be higher. For example, here too, it is possible to select the number that is equivalent to 24 hours or 2 days or more. For example, at a frequency of one connection or data transmission every 60 minutes, this would mean that 24 connections that have not been made are registered by the server before an alarm notification is generated by the server. Identical rules can also apply to the ventilator. Additionally, the ventilator can also be set up to automatically shorten the periods of time after which a connection is supposed to be made after an unsuccessful connection to the server. If for example a connection is supposed to be made after 24 hours, but this is not possible for reasons that are not determined in more detail, the period of time can initially be shortened to 1 to 12 hours. If it is still not possible to make a connection to the server after a specific number of connection attempts, an alarm notification is generated and displayed to the user at the ventilator, for example. The number of unsuccessful connections by the ventilator that lead to an alarm notification can be 1 to 72 unsuccessful connections, for example. The alarm notification can for example also contain, besides the advice that the last connection or connections has/have not taken place, advice that settings on the device may need to be changed and/or the service department should be contacted.

### BRIEF DESCRIPTION OF THE DRAWING

The invention is explained in more detail below using illustrative embodiments with reference to FIG. 1.

FIG. 1 shows an illustrative schematic design of the system comprising a ventilator and a server.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawing making apparent to those of skill in the art how the several forms of the present invention may be embodied in practice.

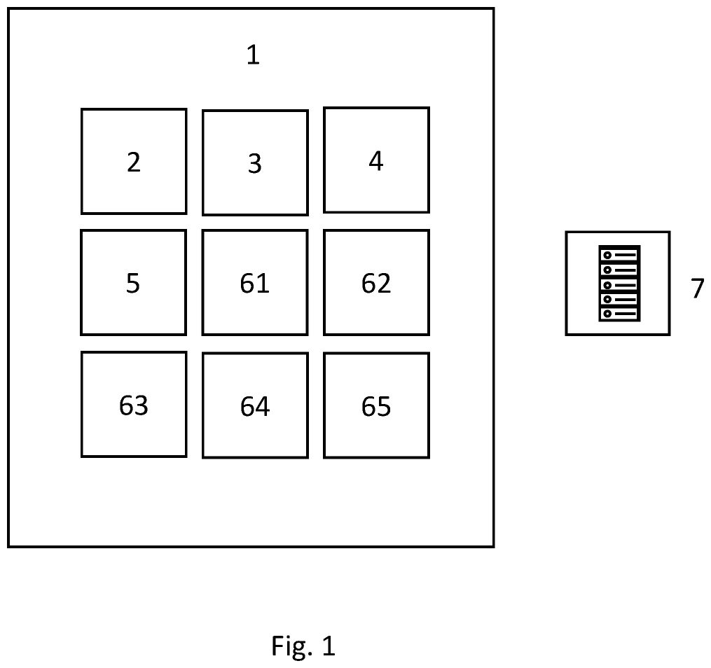

In FIG. 1, the ventilator 1 comprises for example an input unit 2, a communication unit 3, a control unit 4, a user interface 5, a fan and/or valve unit 61, a sensor unit 62, a conditioning unit 63, a storage unit 64 and a monitoring unit 65.

The fan and/or valve unit 61 is designed to produce a respiratory gas airflow for ventilating or treating a patient and to convey said airflow in the direction of the patient if necessary.

The sensor unit 62 is set up to capture measured values, in particular parameters, related to a respiratory flow, a respiratory volume, a respiratory rate, an inhalation and exhalation duration, a respiratory contour, a leakage or a treatment pressure. Optionally, the sensor unit 62 can perform additional measurements for parts or temperature of the respiratory gas or of the blood. The sensor unit 62 transmits the captured measured values to the conditioning unit 63.

The conditioning unit 63 can condition the captured measured values. By way of example, the conditioning unit 63 can perform smoothing, artefact clean-up or down-sampling of the measured values. In some embodiments, the conditioning unit 63 is also in the form of a combined conditioning, calculation and detection unit; alternatively or additionally, these units can also be in the form of separate units. The calculation unit takes the measured values captured by the sensor unit and conditioned by the conditioning unit and calculates signals and/or characteristic quantities, such as for example a mean value, a median, a percentile, a derivative, a frequency distribution, a duration or a proportion of values above or below threshold values. The detection unit is set up to detect events/states such as for example alarms, respiratory dropouts, artefacts, bursts of coughing, oxygen (de)saturations, asynchronous states between the device and the user, inhalation, exhalation or mandatory breaths.

The storage unit 64, which can also be in the form of a buffer-storage unit, for example, stores, or at least buffer-stores, the values/parameters captured by the sensor unit 62 and/or the values, data and/or information conditioned by the conditioning unit 63, inter alia. Buffer-storage means for example that the values, data and/or information are stored up until a transfer and then for example erased or cleared for overwriting.

The monitoring unit 65 detects for example technical problems with the ventilator 1. Technical problems can be for example a low battery level, errors in the electronics, a defective battery, a defective component, a power failure, an incorrectly functioning accessory, an implausible measured value or a departure from a permitted temperature range. The monitoring unit can display or transmit an alarm on the ventilator 1 by way of one of the interfaces in the event of a detected technical problem. In some embodiments, some of these technical problems can also be regarded as serious faults that, other than an appropriate error message on the display (user interface), permit no data transfers.

Additionally, the ventilator 1 comprises for example connections, not shown here, to connect for example a patient interface to the ventilator 1 via a tube.

The input unit 2 is for example set up and designed such that it provides the system or in particular the ventilator 1 with data and/or information. The input unit 2 is connected to the communication unit 3 and the user interface 5 such that the data and/or information input by these very units 3, 5 are made available to the system or the ventilator 1.

The communication unit 3 is used for example primarily to connect the ventilator 1 to the server 7.

The control unit 4 of the ventilator 1 is, by way of illustration, set up to control at least the fan or valve unit 61. In some illustrative embodiments, the control unit 4 is also set up to control the communication unit 3 and/or other or all further parts of the ventilator 1. In some embodiments, a multiplicity of control units can also be used. Generally, the control unit 4 controls the fan or valve unit 61 as predefined by the configurations stored in the storage unit 64 and data ascertained by the sensor unit 62 and possibly conditioned/evaluated by the conditioning unit 63. If the conditioned/evaluated data exceed for example threshold values for respiratory pressures, gas flows and/or respiratory rates and if this is detected by the conditioning unit 63 from the recorded values of the sensor unit 62, the control unit 4 regulates the fan or valve unit 61 accordingly.

The user interface 5 is for example an interface to provide the user/patient at the ventilator with the opportunity to make settings, configurations and inputs and also to display messages (e.g. alarm notifications), information and data (e.g. medical and technical data) if necessary. The user interface 5 can be embodied as a touch-sensitive screen (“touchscreen”), for example, which allows input and display at the same time. The user interface can also be embodied as a screen having additional buttons, switches and/or rotary switches for input. In some illustrative embodiments, the user interface merely consists of one or more control elements for rudimentary control (e.g., switching on/off) and optionally illuminants for status indication. The user interface 5 is, by way of illustration, also set up to input a request to connect the ventilator 1 via the communication unit 3 to the server 7. In some illustrative embodiments, the user interface 5 is an interface for connection to external display and output devices, such as for example a keyboard and an external screen.

The server 7 is, inter alia, set up so that a connection can be made between the communication unit 3 of the ventilator 1 and the server 7. In some illustrative embodiments, the server 7 moreover also has a connected computer or is directly connected to at least one display unit and an input device (e.g. keyboard). Further, the server 7 is for example also designed to store, evaluate, process, forward and/or interpret data and/or information received from the ventilator 1. The server 7 can also transmit information, data and/or configurations to the ventilator 1. The computer and/or the input unit can be used by a user to input inputs, such as e.g., data and information and also configurations, on the server 7, which are then forwarded to the ventilator 1, for example.

To explain the method in more detail by way of illustration, it is assumed that there is already a connection between the ventilator 1 and the server 7. The communication unit 3 is used to transmit data from the ventilator 1 to the server 7. These data contain for example technical and medical data of the ventilator. Technical data can be for example the version of the firmware, settings of the ventilator, fill level and/or wear on consumables, occurrence of technical faults, etc. In some embodiments, these technical data are in particular ascertained by the monitoring unit 65. Medical data comprise for example the times and durations of use of the ventilator, the effectiveness of the ventilator, correct application, and diagnostic and/or treatment-related data, such as for example ventilation pressures, respiratory rate, flow data, temperature of the patient and/or of the respiratory air. Moreover, it is also possible for further medical data, not mentioned here, to be transferred from the ventilator 1 to the server 7.

If all the data requested by the server 7 have been transferred and no further data and/or configurations are supposed to be transmitted from the server 7 to the ventilator 1, the server 7 or a user at the server 7 can decide to terminate the data transfer of at least the medical data and then to also break the connection to the ventilator 1 if necessary. Before the server 7 breaks the connection to the ventilator 1, at least configuration data for communication are transmitted that predefine at least the period of time after which the ventilator 1 is supposed to make the next connection to the server 7. In an alternative or additional embodiment, the ventilator 1 can also automatically determine, for example on the basis of an analysis of the medical data by the conditioning unit, or by input by way of the user interface 5, when it makes the next connection to the server 7. The server 7 and/or also the ventilator 1 decides, for example on the basis of the data or volume of data transmitted from the ventilator 1 to the server 7 during the current connection, or the transmission time, about the period of time before the next connection. In the case of a persistent data transfer of at least medical data, that is to say “live” monitoring of the ventilator 1, the period of time before the next connection can be for example in the range from 1 to 360 minutes or else up to 7 days. For example, the server 7 can use the transmitted medical data to detect what the quality of the ventilation or the condition of the patient in respect of the ventilation and/or treatment is and can then decide whether a further, persistent data transfer and hence also monitoring of the ventilator 1 is needed. The shorter periods of time for the connection and possibly data transfer between the ventilator 1 and the server 7 can be used for example to avoid persistent monitoring, but instead to check the condition of the patient from time to time. By way of example, it is also conceivable, if the condition of the patient and/or use of the ventilator 1 is constantly good, for the periods of time between the connections and possible data transfers to gradually extend until there are for example periods of time of 1 to 7 days between the connections. In the event of a transfer of a data packet, that is to say for example a combination of the data from the period since the last connection, a period of time of 1 to 7 days can be determined, for example. As such, for example a daily or perhaps weekly combination of the data of the ventilator 1 can be requested and received by the server 7. In some embodiments, it is thus possible for example for just the server 7 to be able to terminate the data transfer correctly. An incorrect way of terminating the data transfer would be removal of the power supply of the ventilator 1 or forced deactivation of the data connections of the ventilator 1, for example.

After the connection between the server 7 and the ventilator 1 has been broken, the ventilator 1 waits for the predefined period of time and makes a connection to the server 7 again. In this case, the connection is made only when the ventilator 1 is capable thereof, that is to say for example a power supply, for example by way of the local energy supply, and a data connection are available. It is thus possible to distinguish between connection-compatible modes and non-connection-compatible modes. A connection-compatible mode is for example when an adequate power supply is present, for example by way of a local energy supply or a sufficiently charged battery, and a data connection is available. A non-connection-compatible mode is present for example when the ventilator 1 is in a so-called flight mode, i.e when all data connections are deactivated and are not available. Such a flight mode is switched on inter alia during treatment, in particular during sleep therapy, so that the patient cannot additionally be influenced by radio beams. In some cases, it may also be desirable to allow a data connection during the treatment, for example for “live” monitoring/real-time monitoring. This can be accomplished for example by using a wired data connection or perhaps activating the wireless data connections. In some embodiments, the ventilator 1 moreover automatically detects whether an adequate power supply is present. By way of example, the data connections can be limited or deactivated as soon as the ventilator 1 is isolated from the power grid. Additionally or alternatively, deactivation of the data connections at low battery level (e.g. below 50% or below 30% or below 15%) is also possible. Depending on the quality of the data connection, it is for example also conceivable for the volume of data that is to be transferred to be adapted by the ventilator 1 and/or the server 7, for example fewer data are transmitted in the event of poorer data connections. Moreover, prioritization of the data to be transferred in accordance with the power supply and the data connection is also conceivable.

In some embodiments, there is provision for the connection between the ventilator 1 and the server 7 to be made only by the communication unit 3. A connection can therefore be set up only if the ventilator 1 or a user/patient at the ventilator 1 seeks setup of a connection.

Each fresh connection made to the server 7 results in the ventilator 1 initially always transmitting basic information/device information, such as for example the device type, serial number, firmware version and possibly other device information, to the server 7. In addition to the described step for identifying the ventilator 1, for example further methods for authenticating or protecting the connection can also be performed before beginning the further data transfer between the server 7 and the ventilator 1. In some embodiments, these additional authentication and protection steps can also take place before the transfer of the basic information of the ventilator 1 to the server 7.

The server 7 can use the transferred basic information to identify the ventilator 1 and can individually decide whether and what type of data transfer is requested and specifically which data are supposed to be transferred from the ventilator 1 to the server 7. To that end, the server 7 stores, for example for each individual ventilator (known to the server 7), which data are supposed to be requested, which configurations are supposed to be transmitted to the ventilator and also in which periods of time the ventilator is supposed to connect to the server 7. The server 7 thus compares the identified ventilator 1 against the stored ventilators and transmits the applicable configuration data and a possible request for the transfer of data from the ventilator 1 to the server 7. In accordance with the request, the ventilator 1 begins the data transfer to the server 7 at least until the transfer is terminated by the server 7. During the data transfer, medical and/or technical configuration data and/or configuration data for communication, for example, can moreover also be transmitted to the ventilator 1. Medical configuration data contain for example setting values for the treatment such as e.g ventilation pressures, flow rates, durations and more. Technical configuration data can include for example data that relate to the technical function of the ventilator 1 per se, such as for example a firmware update. The configuration data for communication contain for example settings relating to the volume of data to be transferred, types of data and/or periods of time in which the ventilator 1 is supposed to connect to the server 7. In some embodiments, the server 7 at least always transfers the period of time after which the ventilator 1 is supposed to connect to the server 7 again.

The data query by the server 7, that is to say the requesting of a data transfer of medical data of the ventilator 1, can also be effected automatically by the server 7 in some embodiments.

By way of example, the server 7 can automatically request medical data from the ventilator 1 whenever the ventilator 1 is connected to the server 7 after a period of time, without this needing to be explicitly stipulated by a user at the server 7 beforehand. As described, the server 7 can also use the transferred medical data to automatically stipulate whether further medical data are transferred and/or when the next data transfer is supposed to take place. If the server 7 requests no medical data from the ventilator 1, medical data are also not transferred from the ventilator 1 to the server 7. Whereas, in some embodiments, the server 7 decides about the transfer of medical data entirely automatically, there is provision in some embodiments for a user of the server 7 to be able to manually request at least single data transfers.

In some illustrative embodiments, the data transfer between the server 7 and the ventilator 1 is terminated on the basis of an input on the server 7 and/or an input on the ventilator 1. Moreover, it is conceivable for both inputs on the server 7 and inputs on the ventilator 1 to make a connection independently of the period of time that is set. In some embodiments, the ventilator 1 can also automatically make a connection to the server 7 and begin the data transfer independently of the period of time that is set. This can be the case for example if the measured values and data picked up by the sensor unit 62 and evaluated by the conditioning unit 63 are taken as a basis for detecting a deterioration in the condition of the patient and/or in the quality of the ventilation. That can be detected for example from values above and/or below threshold values in reference to respiratory rate, pressures and/or flows and/or leakage and/or adherence to therapy and/or a deterioration in the synchronism between the device and the patient and/or detection of an intrinsic PEEP of the patient and/or a change in CO2, SpO2, sleep quality, temperature or activity of the patient. The server 7 can also automatically adapt the periods of time in which a connection from the ventilator 1 to the server 7 is supposed to be made on the basis of an analysis of the medical data transferred by the ventilator 1. By way of example, the server 7 can rate a substantially constant quality of the ventilation in such a way that the periods of time after which the ventilator 1 makes a connection to the server 7 and requests a data transfer by the server 7 are increased. A constant quality of the ventilation can be detected by the server 7 for example on the basis of the medical data transferred by the ventilator 1. A constant quality of the ventilation can be detected for example if the parameters associated with breathing, such as pressure, flow and/or rate, exhibit substantially no variations and/or also have no values above or below threshold values. If, on the other hand, variations or deviations in parameters associated with breathing and/or values above or below threshold values are detected, then not only appropriate adaptation of the ventilation parameters by the control unit 4 but also shortening of the periods of time by the server 7 can be prescribed. On the one hand, it is conceivable for the ventilator 1 to evaluate the data by means of the conditioning unit 63 already, so that the points of reference pertaining to the quality of the ventilation are transmitted directly to the server 7 and the server 7 can use these points of reference to decide about the further data transfers. On the other hand, it is also possible, by way of example, for the server 7 itself to rate the medical data of the ventilator 1 in respect of ventilation quality and then to decide about the further data transfers and connections.

If a connection between the ventilator 1 and the server 7 is not made after the period of time that has been set, this is registered by both the ventilator 1 and the server 7. After a specific number of connections that have not been made, the server 7 and/or the ventilator 1 can generate an alarm notification indicating the failed connections. In addition, a connection attempt initiated by the server 7 to the ventilator 1 is also conceivable if a connection has not been set up over a longer period.

In some embodiments, the ventilator 1 automatically shortens the period of time before the next connection to the server 7 if a connection has not been able to be made after the normal period of time. If the original period of time is 24 hours, for example, in order for example to transmit combined use from the past day to the server, but the connection to the server 7 fails after 24 hours, the ventilator 1 attempts to set up a connection again, for example after a shortened period of time of 1 hour, before an alarm notification is generated after a specific number of failed attempts.

If no persistent data transfer, that is to say “live” monitoring or real-time monitoring, takes place, the values and data ascertained by the sensor unit 62 in the monitoring unit 65 and data processed by the conditioning unit 63 are at least buffer-stored in the storage unit 64 and, if requested by the server 7, transferred to the server with the next data transfer.

In some illustrative embodiments, a transfer of medical data takes place in particular only when these data are explicitly requested by the server 7.

It is also conceivable for the transfer to be terminated permanently or perhaps forever. This is advantageous if the ventilator 1 changes user and is reconfigured or adjusted, for example. In this case, it can be advantageous to permanently deregister the device 1 from the server 7. The server 7 can permanently deactivate the ventilator 1. By way of example, it is conceivable for the server 7 to deactivate a SIM card that the device 1 comprises in the device 1.

To sum up, the present invention provides the following items:
1. A method for data transfer within a system, wherein the system comprises at least one ventilator and at least one server, the ventilator comprising at least one input unit and at least one communication unit, the at least one input unit being set up and configured to provide input values and information to the system and the at least one communication unit being set up and configured to make at least one connection to at least one server and to transfer data between the server and the ventilator to thereby transmit data to the server and to receive data from the server, and wherein the data transfer is terminated by the server.
2. The method of item 1, wherein a termination of the data transfer is characterized by the ventilator transferring no further medical data to the server.
3. The method of item 1 or item 2, wherein non-medical data are transferred between the server and the ventilator independently of the data transfer.
4. The method of any one of the preceding items, wherein the server transfers data to the ventilator following the termination of a data transfer by the server.
5. The method of any one of the preceding items, wherein the connection between the server and the ventilator is broken following the termination of a data transfer between the server and the ventilator.
6. The method of any one of the preceding items, wherein a connection between the ventilator and the server is made exclusively by the communication unit, a broken connection resulting in the communication unit connecting to the server in time intervals.
7. The method of any one of the preceding items, wherein initially only device information of the ventilator is transferred to the server when the communication unit of the ventilator has set up a connection to the server again following a time interval, and the server uses transferred device information of the ventilator to identify the ventilator and decides whether and which data are to be transferred from the ventilator to the server.
8. The method of any one of the preceding items, wherein prior to terminating a data transfer and/or prior to breaking a connection the server transmits data concerning a period of time in which the ventilator connects to the server, the ventilator automatically determining a period of time for the next connection to the server on the basis of an analysis of previously transferred data.
9. The method of any one of the preceding items, wherein the server transfers configuration data for communication to the ventilator, the configuration data for communication comprising settings relating to a volume of data that is to be transmitted from the ventilator to the server during a data transfer and/or connection and settings relating to time intervals at which the ventilator is to connect to the server.
10. The method of any one of the preceding items, wherein the ventilator transfers no medical data to the server if these medical data are not requested by the server, a transfer of the data between the ventilator and the server being begun by an action command from the server to the at least one communication unit.
11. The method of any one of the preceding items, wherein the communication unit and/or the server generate or trigger an alarm signal if a specific number of connections of the communication unit to the server that have not taken place or have not been successful in predefined periods of time are registered.
12. The method of any one of the preceding items, wherein a data transfer from the communication unit to the server is automatically terminated by the server on the basis of an analysis of medical data previously transferred by the ventilator, and a connection of the communication unit to the server is automatically broken by the server on the basis of an analysis of the previously transferred data.
13. The method of any one of the preceding items, wherein a data transfer from the communication unit to the server is terminated by the server following an input on the server, and a connection of the communication unit to the server is broken by the server following an input on the server.
14. The method of any one of the preceding items, wherein data are recorded by the ventilator and are stored until the next connection to the server.
15. The method of any one of the preceding items, wherein a broken connection to the server results in the ventilator making a connection to the server on the basis of an analysis of recorded data, and the ventilator initiates a data transfer.
16. The method of any one of the preceding items, wherein the ventilator evaluates and/or combines stored data and wherein these evaluated or combined data are transferred to the server in the course of a new transfer of data, if they are requested by the server.
17. The method of any one of the preceding items, wherein configuration data for communication comprise settings regarding whether data having a high level of detail are to be transferred or whether exclusively rated and/or combined data are to be transferred.
18. The method of any one of the preceding items, wherein a transfer of medical data is prevented after the data transfer has been terminated by the server.
19. A system comprising at least one ventilator and at least one server, wherein the at least one ventilator comprises at least one input unit and at least one communication unit, the at least one input unit being set up and configured to provide input values and information to the system and the at least one communication unit being set up and configured to make at least one connection to the server and to transfer data between the server and the ventilator to thereby transmit data to the server and to receive data from the server, and wherein the data transfer is terminated by the server.
20. A ventilator, wherein the ventilator is capable of being used in a method or a system according to any one of the preceding items.

### LIST OF REFERENCE NUMERALS

1 Ventilator

2 Input unit

3 Communication unit

4 Control unit

5 User interface

7 Server

61 Fan/valve unit

62 Sensor unit

63 Conditioning unit

64 Storage unit

65 unit

What is claimed is:

1. A method for data transfer within a system, wherein the system comprises at least one ventilator and at least one server, the at least one ventilator comprising at least one input unit and at least one communication unit, the at least one input unit being set up and configured to provide input values and information to the system and the at least one communication unit being set up and configured to make at least one connection to at least one server and to transfer data between the at least one server and the at least one ventilator to thereby transmit data to the at least one server and to receive data from the at least one server, and wherein the data transfer is terminated by the at least one server.

2. The method of claim 1, wherein a termination of the data transfer is characterized by the at least one ventilator transferring no further medical data to the at least one server.

3. The method of claim 1, wherein non-medical data are transferred between the at least one server and the at least one ventilator independently of the data transfer.

4. The method of claim 1, wherein the at least one server transfers data to the at least one ventilator following a termination of a data transfer by the at least one server.

5. The method of claim 1, wherein a connection between the at least one server and the at least one ventilator is broken following termination of a data transfer between the at least one server and the at least one ventilator.

6. The method of claim 1, wherein a connection between the at least one ventilator and the at least one server is made exclusively by the at least one communication unit, a broken connection resulting in the at least one communication unit connecting to the at least one server in time intervals.

7. The method of claim 1, wherein initially only device information of the at least one ventilator is transferred to the at least one server when the at least one communication unit of the at least one ventilator has set up a connection to the at least one server again following a time interval, and the at least one server uses transferred device information of the at least one ventilator to identify the ventilator and decides whether and which data are to be transferred from the at least one ventilator to the at least one server.

8. The method of claim 1, wherein prior to terminating a data transfer and/or prior to breaking a connection the at least one server transmits data concerning a period of time in which the at least one ventilator connects to the at least one server, the at least one ventilator automatically determining a period of time for the next connection to the at least one server on the basis of an analysis of previously transferred data.

9. The method of claim 1, wherein the at least one server transfers configuration data for communication to the at least one ventilator, the configuration data for communication comprising settings relating to a volume of data that is to be transmitted from the at least one ventilator to the at least one server during a data transfer and/or connection and settings relating to time intervals at which the at least one ventilator is to connect to the at least one server.

10. The method of claim 1, wherein the at least one ventilator transfers no medical data to the at least one server if these medical data are not requested by the at least one server, a transfer of the data between the at least one ventilator and the at least one server being begun by an action command from the at least one server to the at least one communication unit.

11. The method of claim 1, wherein the at least one communication unit and/or the at least one server generate or trigger an alarm signal if a specific number of connections of the at least one communication unit to the at least one server that have not taken place or have not been successful in predefined periods of time are registered.

12. The method of claim 1, wherein a data transfer from the at least one communication unit to the at least one server is automatically terminated by the at least one server on the basis of an analysis of medical data previously transferred by the at least one ventilator, and a connection of the at least one communication unit to the at least one server is automatically broken by the at least one server on the basis of an analysis of the previously transferred data.

13. The method of claim 1, wherein a data transfer from the at least one communication unit to the at least one server is terminated by the at least one server following an input on the at least one server, and a connection of the at least one communication unit to the at least one server is broken by the server following an input on the server.

14. The method of claim 1, wherein data are recorded by the at least one ventilator and are stored until the next connection to the at least one server.

15. The method of claim 1, wherein a broken connection to the at least one server results in the at least one ventilator making a connection to the at least one server on the basis of an analysis of recorded data, and the at least one ventilator initiates a data transfer.

16. The method of claim 1, wherein the at least one ventilator evaluates and/or combines stored data and wherein these evaluated or combined data are transferred to the at least one server in the course of a new transfer of data, if they are requested by the at least one server.

17. The method of claim 1, wherein configuration data for communication comprise settings regarding whether data having a high level of detail are to be transferred or whether exclusively rated and/or combined data are to be transferred.

18. The method of claim 1, wherein a transfer of medical data is prevented after the data transfer has been terminated by the at least one server.

19. A system comprising at least one ventilator and at least one server, wherein the at least one ventilator comprises at least one input unit and at least one communication unit, the at least one input unit being set up and configured to provide input values and information to the system and the at least one communication unit being set up and configured to make at least one connection to the at least one server and to transfer data between the at least one server and the at least one ventilator to thereby transmit data to the at least one server and to receive data from the at least one server, and wherein the data transfer is terminated by the at least one server.

20. A ventilator, wherein the ventilator is capable of being used in a method according to claim 1.
